(19) United States
(12) Patent Application Publication
(10) Pub. No.: US 2009/0133582 A1
(43) Pub. Date: May 28, 2009

(54) Filter Apparatus

(76) Inventor: Malcolm Snowball, Essex (GB)

Correspondence Address: GORDON & JACOBSON, P.C., 60 LONG RIDGE ROAD, SUITE 407, STAMFORD, CT, 06902, US

(21) Appl. No.: 11/997,652

(22) PCT Filed: Jul. 26, 2006
(86) PCT No.: PCT/GB06/50223

§ 371 Date: Aug. 5, 2008

(30) Foreign Application Priority Data
Aug. 3, 2005 (GB) 0515940.5

(51) Int. Cl. F24F 3/16; A61L 9/20

(52) U.S. Cl. 96/224

(57) ABSTRACT

A ventilation system has a filter apparatus which comprises a porous filter media 10 arranged to trap micro-organisms contained in a fluid flow along a duct 14 of the apparatus, and a lamp 13 for irradiating the filter media 10 with ultraviolet light, the filter media 10 being formed of a fluroplastics material which is substantially transparent to the ultraviolet light so that micro-organisms trapped inside the pores of the filter are irradiated and killed by the ultraviolet light.

The ventilation system alternatively or additionally comprises a filter apparatus which comprises a porous filter media 40 formed of an electrically conductive material arranged to trap micro-organisms contained in a fluid flow along a duct 42 of the apparatus, and a coil 25 for irradiating the filter media 40 with electromagnetic radiation so as to heat the filter media 45 and thereby kill any micro-organisms trapped by the filter media 40.

### BACKGROUND OF THE INVENTION

1. Field of the Invention

This invention relates to a filter apparatus for treating fluids and more particularly but not solely to an apparatus for filtering and disinfecting air.

2. Related Background Art

There are usually three non-chemical approaches to controlling biological contamination in air, these approaches involving the use of filters, UV disinfectors or heat.

It is well known that high intensity UV light in the wavebands 220 nm-280 nm, which are called the germicidal wavelengths, has germicidal properties that can kill all known micro-organisms and therefore should be the ideal technology for disinfecting air. This is the case for some applications but other applications highlight shortcomings in this technology.

Micro-organisms have a disparate UV dose to kill ratio depending on the type of micro-organism to be controlled. For example the bacteria legionella has a 99.9% kill with an applied UV dose of 6-9 mj/cm², where some of the mould spores have a 99.9% kill with an applied UV dose of 220-330 mj/cm². In air systems where mould spores or indeed bacterial spores need to be controlled using UV technology the only way to do this is with very powerful UV systems. These systems are very energy inefficient and are therefore expensive to run.

It is also well known that biological contamination of air can be successfully treated by applying filtration to the air for example using a HEPA high efficiency particulate air filter, which will filter 99.97% of all particles 0.3 microns and above thereby capturing virtually all bacteria and mould spores. The HEPA filter is used because of its good filtration performance but, unfortunately there are several problems associated with the use of these filters.

HEPA filters and indeed all filters with a guaranteed pore size of 0.3 micron or less will efficiently filter out all bacteria, bacterial spores and mould spores but can be a hazardous source of infection in their own right. The barrier filter action of the HEPA filter on the micro-organisms most of which could be pathogenic causes a continual build up of micro-organisms in the filter media and this, together with the fact that these micro-organisms will further increase their numbers by breeding in the filter, turns the filter into a significant biological hazard. Furthermore, the disposal of such a filter needs strict control but if such a filter bursts or leaks then it has the ability to infect the air passing through it and hence the general public at large.

Another significant problem is that whilst such filters will efficiently filter out all bacteria, bacterial spores and mould spores they are completely ineffective against viruses.

These filters are manufactured using several different processes but all achieve the same objective providing a barrier in the form of a matrix of fibrous material usually of sub-micron size, which is constructed in a manner to produce pores of a specific size. The fluid to be treated passes through the pores and the contaminants are size excluded from passing through the pores because the pore size is too small for the contaminant to pass.

The third method of non-chemical disinfection is by heat, whereby the micro-organisms are subjected to temperatures which kill or inactivate them.

I have now devised a filter apparatus which is relatively simple and inexpensive in construction yet is able to effectively capture and kill micro-organisms and viruses contained in a fluid flow.

### SUMMARY OF THE INVENTION

In accordance with this invention, as seen from a first aspect, there is provided a filter apparatus comprising a porous filter media arranged to trap micro-organisms contained in a fluid flow through the apparatus and means for irradiating the filter media with ultraviolet light, wherein the filter media is formed of a material which is substantially transparent to said ultraviolet light.

In use, the filter is irradiated with ultraviolet light in the germicidal range to kill the trapped micro-organisms. The kill efficiency for a specific micro-organism is directly related to the intensity of the germicidal radiation multiplied by the time that the radiation illuminates the micro-organism.

Dose “D”=Intensity “I”×Exposure time “t”

The micro-organism is immobilized by the filter therefore it is irradiated for considerable lengths of time; this means that the radiation source does not need to be powerful and indeed can be quite small. For example assume that the target micro-organism is a mould spore which needs a dose of UV radiation of 220 mj/cm² to achieve a 4 log kill. Depending upon the distance from the filter surface, an 18 watt UV lamp will produce radiation intensity through the filter of 4 mW/cm² which will result in the 4 log kill dose being reached in 55 seconds. With this technique not only is the surface of the filter kept disinfected but also the interior is kept substantially biologically disinfected.

In order to overcome the problem of shading of the UV radiation due to debris in the filter, the filter is preferably formed fibres or cells, which are substantially transparent to the germicidal wavelengths. The fibres or cell walls act as light guides which transport the germicidal wavelengths around the whole of the filter. These fibres or cell walls are preferably not perfect light guides and the UV light leaks from the fibre due to scattering or incomplete reflection, thereby providing illumination in all parts of the filter and hence irradiating all micro-organisms in caught in the filter.

Preferably the filter is shaped to maximize the surface area of the media providing good flow with low pressure drop. Preferably the filter is constructed as a High Efficiency Particulate Air (HEPA) Filter.

Preferably the filter is formed of a material which is substantially transparent to UV radiation in the wavelength range 200 nm-300 nm. Preferably the filter material is of the fluorocarbon family such as Polytetraflouroethylene (PTFE) or the polyethylene family of plastics or woven quartz filaments or any other material which is substantially or partially transparent to the germicidal wavelengths. A preferred material is Teflon FEP. Preferably the filter is constructed using the HEPA design providing a matrix of pores which provides barrier filtration with depth for good particulate holding qualities.

Preferably the filter material is fibrous and the fibre diameter is sub-micron such that when it is constructed into a depth filter it produces pores of substantially constant size. Preferably the pore size is in the HEPA range of 0.3 microns or smaller.

Means are provided to support the filter by a structure which allows the filter to substantially hold its shape when air is passing through it. Preferably the support structure is designed to include means to guide or duct all of the air through the filter, such that it passes through the filter material without bypassing the filter.

The fluid treatment apparatus as described is positioned such that fluid or more particularly air, which is biologically contaminated, is caused to flow through the filter. The air flows through the pores of the filter and the biological contamination is size excluded and retained in the filter. The UV germicidal radiation from the UV lamps placed to irradiate the entire filter and substantially penetrates the depth of the filter. The radiation is also carried by the filter fibres and is distributed throughout the whole body of the filter. This radiation is leaked into every part of the filter by natural scattering from the fibres which are imperfect light guides. Any biological contamination is irradiated for long periods of time which creates very high UV radiation doses resulting in deactivation of the micro-organisms causing the biological contamination.

Any viruses which pass through the pores of the filter are irradiated by the germicidal wavelengths as they leave the filter.

The filter efficiency may be improved by introducing an electrostatic charge to the filter material, either by material selection or by the use of an external electrostatic field.

Also in accordance with this invention, as seen from the first aspect, there is provided a ventilation system comprising an air flow duct and a filter apparatus as hereinbefore described mounted in said flow duct.

Preferably the means for irradiating the filter media with ultraviolet light is mounted downstream of the filter media.

In accordance with this invention, as seen from a second aspect, there is provided a filter apparatus comprising a porous filter media arranged to trap micro-organisms contained in a fluid flow through the apparatus and means for irradiating the filter media with electromagnetic radiation, wherein the filter media is formed of an electrically conductive material which is heated by said radiation.

The heated filter pasteurises and kills any trapped micro-organisms.

Preferably the filter is formed of 403 grade stainless iron, mild steel or any other suitable metal able to be heated by induction heating techniques.

Preferably the filter is shaped to maximize the surface area of the media providing good flow with low pressure drop. Preferably the filter is constructed as a High Efficiency Particulate Air (HEPA) Filter having a matrix of pores which provides barrier filtration with depth for good particulate holding qualities. Preferably the filter material is such that when it is constructed into a filter it produces pores of substantially constant size. Preferably the pore size is in the HEPA range of 0.3 microns or smaller. The material may be woven, spun into metal wool or created by sintering techniques using metal powder compressed into shape and then sintered to form a regular porous material.

Means are provided to support the filter by a structure which allows the filter to substantially hold its shape when air is passing through it. Preferably the support structure is designed to include means to guide or duct all of the air through the filter, such that it passes through the filter material without bypassing the filter.

Means are provided to irradiate the filter with electromagnetic radiation. Preferably the electromagnetic radiation is in the form of a high frequency magnetic field placed in close proximity with the filter. Preferably the source of the electromagnetic radiation is in the form of a coil energized with high frequency current, which is placed such that the filter is in the electromagnetic field. Under these conditions the filter material will have eddy currents induced substantially throughout its bulk material. The eddy currents travel in a circular path around each magnetic line of force and therefore through the filter material. The filter material not being an ideal conductor of electrical current has electrical resistance and therefore will heat up according to the law:

Power P=I²×R=watts.

Preferably means are provided to monitor the filter temperature to ensure maximum disinfection with minimum power usage. The heat disinfection effect can be actioned on an as is required basis, a time basis or continuously depending upon the application.

The fluid treatment apparatus as described is positioned such that fluid or more particularly air, which is biologically contaminated, is caused to flow through the filter. The air flows through the pores of the filter and the micro-organisms are size excluded and retained in the filter. A coil is placed in front of the filter in close proximity to the filter. Means are provided to energize the coil with a high frequency signal from a suitable generator, which produces a corresponding high frequency electromagnetic field. The electromagnetic field from the coil is positioned to substantially cover the entire surface of the filter and penetrate the whole depth of the filter. The filter is a good conductor of heat so as the filter material heats up the heat is conducted to all parts of the filter any biological contamination is heated for long periods of time. This results in a very effective pasteurization process in which all of the micro-organisms causing the biological contamination including viruses are killed or deactivated.

Also in accordance with this invention, as seen from the second aspect, there is provided a ventilation system comprising an air flow duct and a filter apparatus as hereinbefore described mounted in said flow duct.

Preferably the means for irradiating the filter media with electromagnetic radiation is mounted downstream of the filter media.

### BRIEF DESCRIPTION OF THE DRAWINGS

Embodiments of the invention will now be described by ways of examples only and with reference to the accompanying drawings, in which:

FIG. 1 is an isometric view of an embodiment of air filter apparatus in accordance with the first aspect of this invention, when mounted inside an air duct;

FIG. 2 is an isometric view of an alternative embodiment of air filter apparatus in accordance with the first aspect of this invention, when mounted inside an air duct;

FIG. 3 is an enlarged view showing the pores of the filter media of the filter assembly of FIG. 1 or FIG. 2; and

FIG. 4 is an isometric view of an embodiment of air filter apparatus in accordance with the second aspect of this invention, when mounted inside an air duct.

### DETAILED DESCRIPTION OF THE PREFERRED EMBODIMENTS

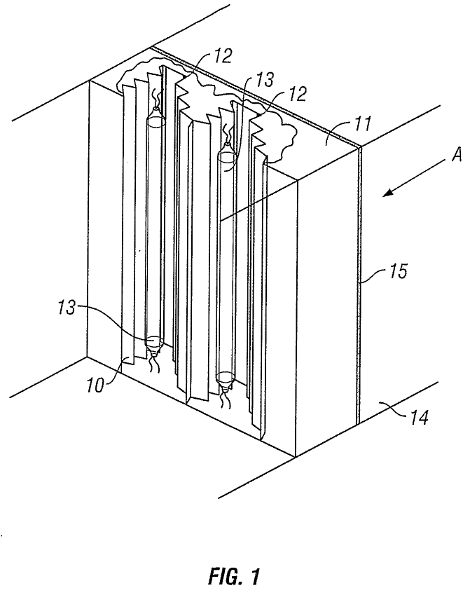

Referring to FIG. 1 of the drawings, there is shown a pleated filter media 10 mounted in a support frame 11. The filter 10 is supported in the support frame 11 by support ribs 12 such that substantially hold it in shape when air passes through it. The media 10 is pleated to provide a large surface area and low pressure drop. Lamps 13 which radiate most or part of their output in the ultra violet wavelengths are provided to irradiate the filter media 10 and are positioned adjacent thereto. The lamps 13 are elongate and radiate most or part of their output in the germicidal wavelengths 220 nm to 280 nm. The lamps 13 are placed such that substantially the entire downstream surface of the filter media 10 is directly irradiated.

The lamps 13 can be positioned in any aspect in the plane parallel to the filter media 10: the diagram shows the lamps 13 extending parallel to the pleated filter media 10. The lamps 13 are disposed on the exhaust side of the filter media 10 therefore allowing the filter media 10 to keep the lamps 13 clean, this also allows the lamps 13 to be changed without compromising the integrity of the disinfection action.

In this configuration, the system is less sensitive to lamp failure provided that the lamps 13 are changed on a regular basis.

The whole filter assembly is placed in a rectilinear duct 14 and sealed to the duct walls by a resilient seal 15, such that any air passing along the duct is forced to pass through the filter media 10. The filter pores of the media 10 are sized to size—exclude any target micro-organisms carried by the airflow A and trap them in the filter media 10. The trapped micro-organisms are irradiated by the lamps 13 through the filter media 10 and are killed or deactivated. Because of the long retention times associated with this system and the fact that the fibres of the filter media 10 act as an imperfect light guide scattering the radiation throughout the filter media 10, any solid particulate which would normally act as a block to the radiation is circumnavigated by the light guide effect. Effectively the entire filter volume of the filter media 10 receives radiation for extremely long periods of time and therefore very high doses of radiation are delivered.

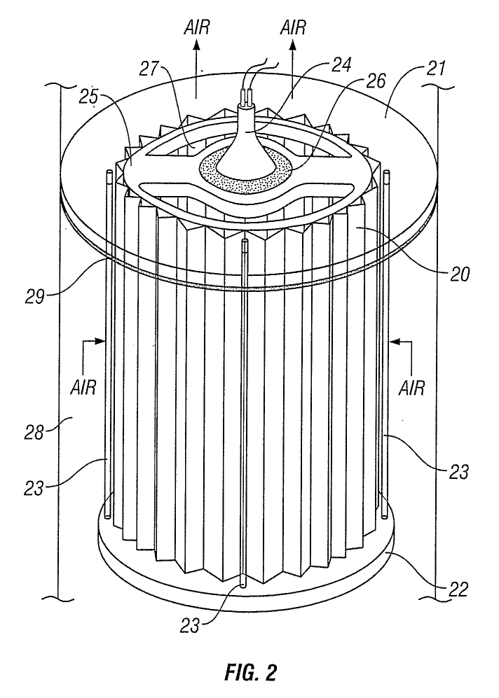

Referring to FIG. 2 of the drawings, there is shown a tubular pleated filter media 20 mounted in a support frame having upper and lower frame members 21,22. The filter media 20 is supported in the frame by axially extending support ribs 23 arranged to substantially maintain the shape of the filter media 20 when air passes through it. The pleats serve to maximize the surface area of the filter media 20 and to provide a low pressure drop. Means are provided to irradiate the filter media 20 in the form of a lamp 24 which radiates most or part of its output in the ultra violet wavelengths and is placed adjacent to the filter media 20. The lamp 24 is elongate and radiates most or part of its output in the germicidal wavelengths 220 nm to 280 nm. The lamp 24 extends along the longitudinal central axis of the filter media 20, such that the entire filter media 20 is irradiated.

The lamp 24 is positioned in the centre of the tubular filter media 20 via a clamp 25 which acts as an anchor for the filter media 20 and provides a base for a lamp seal 26 the combination of which effectively position the lamp 24 and the filter media 20 at the upper end of the filter media 20. The clamp 25 has exhaust slots 27 to allow the air to exhaust into the duct past the filter media 20. A corresponding clamp (not shown) also acts as an anchor for the filter media 20 and provides a base for a lower lamp seal (not shown), the combination of which effectively position the lamp 24 and the filter media 20 at the lower end of the filter media 20.

In use, airflows radially inwardly through the tubular pleated filter media 20 and axially upwardly through the slots 27 in the clamp 25. The lamp 24 is positioned on the exhaust side of the filter media 20 therefore allowing the filter media 20 to keep the lamp 24 clean, this also allows the lamp 24 to be changed without compromising the integrity of the disinfection action. In this configuration, the system is less sensitive to lamp failure provided that the lamp 24 is changed on a regular basis.

The whole filter assembly is placed in a tubular duct 28 and sealed to the duct walls by a resilient seal 29 such that any air passing along the duct is forced to pass through the filter assembly. Preferably the filter pores are sized to size—exclude any target micro-organisms carried by the air and trap them in the filter media 20. The trapped micro-organisms are irradiated by the lamp 24 through the filter media 20 and killed or deactivated. Because of the long retention times associated with this system and the fact that the fibres of the filter media 20 act as an imperfect light guide scattering the radiation throughout the filter, then any solid particulate such as dust which would normally act as a block to the radiation is circumnavigated by the light guide effect. Effectively the entire filter media 20 receives radiation for extremely long periods of time therefore a very high dose of UV radiation is produced.

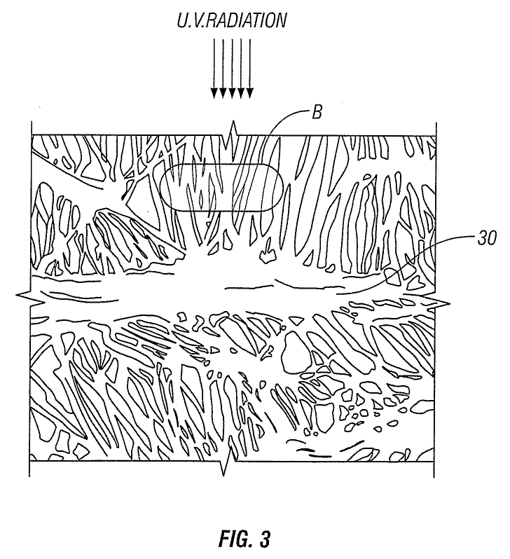

FIG. 3 shows a bacterium B which has been size-excluded and trapped by the fibres 30 of a melt-blown filter media 20 as is being irradiated with UV radiation at the germicidal wavelengths.

The invention described with reference to FIGS. 1 & 2 can have many variations, for example the filter material could be made like a cartridge filter so that it could be quickly attached and unattached to a base which supported a lamp, when assembled the lamp being positioned inside the filter cartridge therefore making the filter cartridge easily changed.

The duct could have a wall section which is substantially transparent to the germicidal wavelengths and the UV radiation could irradiate the filter material from outside of the duct.

The filter can have many shapes and lamp positions/configurations to accomplish the invention which those skilled in the art would be able to perfect.

There are applications where the filter material must be very robust e.g. military, space industries or high integrity biological safety applications. The usual material selection for these applications is some form of metal.

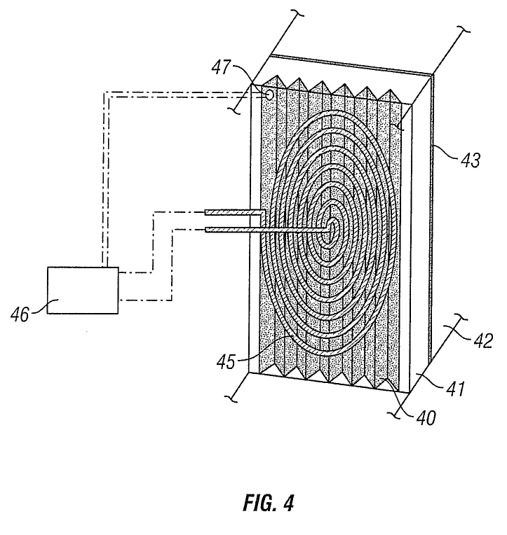

Referring to FIG. 4 of the drawings, there is shown a filter media 40 mounted in a support frame 41. The filter media 40 is made from a material which can be heated by induction heating techniques as described previously. Preferably the material is sintered stainless iron type 430, stainless steel, mild steel or any other suitable material which can be inductively heated. The filter media 40 is fixed into the support frame 41 such that it forms a pleated wall for maximum surface area and to strengthen the filter media 40. The pleated wall can be formed either by taking a sheet of sintered material, of a material which can be inductively heated and folding it into the pleated shape, or alternatively using a plurality of sintered material strips of the same material and bonding them into the pleated shape. The whole filter assembly is placed in a rectilinear duct 42 and sealed to the duct walls by a resilient seal 43 such that any air passing along the duct is forced to pass through the filter media 40. Means are provided to irradiate the filter material with an electromagnetic field in the form of a coil 45 energized with high frequency current.

The coil 45 is open wound and is supported on a suitable frame (not shown) placed adjacent to and in close proximity to the filter media 40. The coil 45 is open wound so that it imposes a minimal pressure drop behind the filter media 40. The coil 45 is energized with a high frequency current from a suitable high frequency current generator 46 and consequentially produces a high frequency electromagnetic field. The filter media 40 is positioned such that it is in the electromagnetic field, the filter media 40 being made of a material which is able to be heated by induction heating techniques immediately heats up as described previously. Means are provided to measure the temperature of the filter in the form of a temperature sensing device 47. The signal generated by this device is fed back to the current generator which in turn uses the information to regulate the temperature of the filter by regulating the HF current into the coil 45. The control unit 46 then holds the filter media 40 at the correct temperature for the appropriate time for complete pasteurization of the filter media 40.

While the preferred embodiments of the invention have been shown and described, it will be understood by those skilled in the art that changes of modifications may be made thereto without departing from the true spirit and scope of the invention.

1. A filter apparatus comprising a porous filter media arranged to trap micro-organisms contained in a fluid flow through the apparatus and means for irradiating the filter media with ultraviolet light, wherein the filter media is formed of a material which is substantially transparent to said ultraviolet light.

2. A filter apparatus as claimed in claim 1, in which the filter media is formed fibrous or cellular material which is substantially transparent to light having a wavelength or wavelengths in the range of 220 nm-300 nm.

3. A filter apparatus as claimed in claim 2, in which the filter media is formed fibrous or cellular material arranged to allow the ultraviolet light to leak therefrom.

4. A filter apparatus as claimed in claim 1, in which the filter is shaped to maximize the surface area of the media providing good flow with low pressure drop.

5. A filter apparatus as claimed in claim 1, in which the filter media is formed as a High Efficiency Particulate Air (HEPA) Filter.

6. A filter apparatus as claimed in claim 1, in which said material is of the fluorocarbon family of materials.

7. A filter apparatus as claimed in claim 6, in which said material is a fluoropolymer.

8. A filter apparatus as claimed in claim 1, in which said material is fibrous, the fibre diameter being sub-micron.

9. A filter apparatus as claimed in claim 1, in which the filter media comprises pores of substantially uniform size.

10. A filter apparatus as claimed in claim 1, in which the filter media comprises pores having a size of 0.3 microns or smaller.

11. A filter apparatus as claimed in claim 1, comprising means for introducing an electrostatic charge to the filter media.

12. A filter apparatus as claimed in claim 1, in which said irradiating means is positioned downstream of said filter media.

13. A filter apparatus as claimed in claim 1, in which said filter media is tubular, said irradiating means being positioned inside a space defined by said tubular media.

14. A filter apparatus as claimed in claim 1, in which means are provided for heating said filter media.

15. A filter apparatus as claimed in claim 1, comprising means for irradiating the filter media with electromagnetic radiation, the filter media being formed of an electrically conductive material which is heated by said radiation.

16. A ventilation system comprising an air flow duct and a filter apparatus as claimed in claim 1.

17. A ventilation system as claimed in claim 16, in which means for irradiating the filter media with ultraviolet light are mounted downstream of the filter media in said duct.

18. A ventilation system as claimed in claim 16, in which means for irradiating the filter media are arranged externally of said on the opposite side of a transparent wall portion of the duct.

19. A filter apparatus comprising a porous filter media arranged to trap micro-organisms contained in a fluid flow through the apparatus and means for irradiating the filter media with electromagnetic radiation, wherein the filter media is formed of an electrically conductive material which is heated by said radiation.

20. A filter apparatus as claimed in claim 19, in which the filter media is formed of 403 grade stainless iron, mild steel or any other suitable metal able to be heated by induction heating techniques.

21. A filter apparatus as claimed in claims 19, in which the means for irradiating the filter media comprises a coil and means for energising the coil with high frequency current, the coil being mounted such that the filter media is in said electromagnetic field.

22. A ventilation system comprising an air flow duct and a filter apparatus as claimed in claim 19.

23. A ventilation system as claimed in claim 22, in which means for irradiating the filter media with electromagnetic radiation is mounted downstream of the filter media in said duct.

24. A ventilation system as claimed in claim 22, comprising a filter apparatus as claimed in claim 1 mounted in series in the duct with a filter apparatus as claimed in claim 19.
